(19) United States
(12) Patent Application Publication
(10) Pub. No.: US 2001/0025183 A1
(43) Pub. Date: Sep. 27, 2001

(54) Methods and apparatuses for maintaining a trajectory in sterotaxi for tracking a target inside a body

(76) Inventor: Ramin Shahidi, San Francisco, CA (US)

Correspondence Address: IOTA PI LAW GROUP, 350 CAMBRIDGE AVENUE SUITE 250, P O BOX 60850, PALO ALTO, CA, 94306-0850, US

(21) Appl. No.: 09/792,485

(22) Filed: Feb. 23, 2001

Related U.S. Application Data
(63) Continuation of application No. 60/185,036, filed on Feb. 25, 2000.

(51) Int. Cl. A61B 19/00

(52) U.S. Cl. 606/130

(57) ABSTRACT

An apparatus and method for adjusting the orientation of a surgical viewing instrument, which may be used to view a patient target site and any intervening tissue from outside the body, as the position of the instrument is changed by a user. The instrument is attached to a robotic arm assembly and is movable by both the user and the robot. As the user moves the instrument to a different position, the robot automatically corrects the orientation of the instrument to maintain a viewing trajectory defined by the axis of the instrument and a target coordinate in the patient target site. In another aspect there is an apparatus and method for using a surgical robot and attached ultrasound probe to track a moving target in a patient's body. The ultrasound probe has a pressure sensor in its tip, which is maintained in contact with a tissue surface at a specific location at a constant pressure. Subject to this constraint, the robot is directed to adjust the orientation of the probe, as the target point moves, to maintain the axis of the probe in line with the target point.

### FIELD OF THE INVENTION

The present invention generally relates to image-guided, robotic-assisted surgical techniques. More specifically, the invention relates to an apparatus and method for orienting the axis of an instrument on a processor-controlled robotic arm toward a target point in the patient's body to enable a user to find an optimal approach to the target point, as the robotic arm is freely moved in space. The invention also relates to an apparatus and method for tracking a moving indicator inside the body using a processor-controlled robotic arm with a distal-end probe whose tip is held in constant contact with a body surface while the axis of the probe is aligned with the moving indicator. The invention also relates to a processor-readable medium embodying a program of instructions (i.e., software) for implementing each of the methods.

### BACKGROUND OF THE INVENTION

In the past several years, the field of image-guided surgery has experienced rapid progress. Recent developments in computation technology allow surgeons to visualize real-time three-dimensional images of a patient target site during surgery. These techniques also allow the surgeon to decide where to position the surgical instrument(s). Such guidance information has the potential to enable surgeons to achieve more successful clinical outcomes with the added benefits of reduced complications, pain and trauma to the patient.

In one form, image-guided surgery generally involves: (1) acquiring 2-D images of internal anatomical structures of interest, i.e., of a patient target site; (2) reformatting a 2-D image or reconstructing a 3-D image based on the acquired 2-D images; (3) manipulating the images; (4) registering the patient's physical anatomy to the images; (5) targeting a site of interest in the patient; and (6) navigating to that site.

Typically, the acquired 2-D images are reformatted to generate two additional sets of 2-D images. One of the sets of images is parallel to a first plane defined by two of the three axes in a 3-D coordinate system, say, the xy-plane; a second set is parallel to, say, the xz-plane; and a third set is parallel to, say, the yz-plane.

The registration process is the point-for-point mapping of one space (e.g., the physical space in which the patient resides) to another space (e.g., the image space in which the patient is viewed). Registration between the patient and the image provides a basis by which a medical instrument can be tracked in the images as it is moved within the operating field during surgery.

A 3-D localizer is used to track the medical instrument relative to the internal structures of the patient as it is navigated in and around the patient target site during surgery. Images of the target site are displayed on a computer monitor to assist the user (e.g., a surgeon) in navigating to the target site. Tracking may be based on, for example, the known mathematics of “triangulation.”

Further details regarding techniques involved in image-guided surgery are disclosed in international application, publication no.: WO 99/00052, publication date: Jan. 7, 1999. The contents of this application are incorporated herein by reference.

For certain surgical tasks, it may not be possible to accurately achieve the preoperative objectives using only image-based navigational guidance. For such tasks, it may be appropriate to incorporate a robotic or computer-controlled mechanical arm into the image-based navigational system to assist in certain surgical procedures where precision and steadiness is important. For example, robots have been used in orthopedic surgery to precisely position and operate a high-speed pneumatic cutter to remove bone within a patient's femoral canal.

However, one useful technique that conventional image-guided, robotic-assisted surgery does not provide is a technique for determining an optimal point of entry of a surgical tool to be used by a surgeon in accessing a target site within the patient's body, by enabling the surgeon to move a viewing instrument in space while a robot to which the instrument is attached enforces the instrument's orientation in the direction of a target point, thereby enabling the surgeon to view the target site and any intervening tissue along the axis of the instrument, as it is moved.

Another useful technique that conventional image-guided, robotic-assisted surgery does not provide is a technique for tracking a moving target in the patient's body using a robot-held probe whose orientation is enforced in the direction of the target while the probe tip is held at a constant pressure against a surface of the body.

### SUMMARY OF THE INVENTION

The present invention overcomes these problems by providing apparatuses and methods for accomplishing these techniques.

In one aspect, the invention involves a device for determining the optimal point of entry of a surgical tool adapted for use by a surgeon in accessing a target site within a patient's body. The device includes an articulated mechanical arm, such as multi-segmented robotic arm, having or accommodating a distal-end pointer, and a tracking controller that tracks the position and orientation of the pointer with respect to a predetermined target coordinate. An imaging device in communication with the tracking controller generates an image of the target site and intervening tissue as seen from a selected point outside of the body, along a line between that point and the target point coordinate. An actuator, in communication with the tracking controller, adjusts the position of the mechanical arm so as to orient the axis of the pointer in the direction of the target point coordinate, as the pointer is moved in space to a selected position outside the body, such that the user can approach the target site, or view the target site and intervening tissue, along a trajectory from the selected position to the target point coordinate.

Preferably, the imaging device constructs an image of the target site using previously obtained scan data, and the predetermined target coordinate is assigned using the constructed image.

Once the optimal point of entry is determined, the pointer can be replaced with a surgical tool to enter the patient's target site along the established trajectory.

In another aspect, the invention involves a method for maintaining a trajectory toward a target site and for viewing any intervening tissue along the trajectory, as defined by the axis of a viewing instrument and a target coordinate in the target site, while the instrument is moved in space. The method comprises acquiring scans of the patient; using the acquired scans to construct an image of the patient target site; assigning the target coordinate on the constructed image; correlating an image coordinate system with an instrument coordinate system; and controlling the orientation of the instrument to maintain the defined trajectory, as the instrument is moved in space outside the body.

This method may be implemented using a program of instructions (e.g., software) that is embodied on a processor-readable medium and that is executed by a processor.

In a further aspect, the invention involves a device for maintaining a trajectory between a tip of an instrument and a moving target in a patient's body. The device includes an articulated mechanical arm having or accommodating a distal-end instrument having a tip that has or accommodates a force contact sensor, and a tracking mechanism for tracking the position and orientation of the instrument with respect to coordinates of the moving target. A processor in communication with the tracking mechanism calculates and updates the coordinates of the moving target. An actuator, in communication with the tracking mechanism, adjusts the orientation of the mechanical arm, while maintaining a constant pressure between the instrument tip and a surface of the body, so as to maintain the trajectory between the tip of the instrument in the direction of the moving target.

In still another aspect, the invention involves a method for maintaining a trajectory between a tip of an instrument and a moving target in a patient's body using a robot-held instrument. The method comprises acquiring scans of the patient; using the acquired scans to construct an image of the patient target site; assigning the target coordinate on the constructed image; and controlling the orientation of the instrument to maintain a trajectory defined by the axis of the probe and a point on the moving target, while maintaining the tip of the instrument at a fixed location against a tissue surface at a constant pressure, as the instrument is moved in space outside the body.

This method may also be implemented using a program of instructions (e.g., software) that is embodied on a processor-readable medium and that is executed by a processor.

### BRIEF DESCRIPTION OF THE FIGURES

FIG. 1 is a partially perspective, partially schematic view of an image-guided, robotic-assisted surgery system constructed in accordance with embodiments of the invention.

FIG. 2 is a flow chart illustrating a general mode of operation in accordance with embodiments of the present invention.

FIG. 3 is a schematic view of the robotic assembly and target point, showing the robot in different positions with the pointer's orientation directed at the target point, in accordance with a first embodiment of the invention.

FIG. 4 is a flow chart illustrating the tracking process, according to a first embodiment of the invention.

FIG. 5 is a schematic view of the robotic assembly, target point and tissue surface, showing the robot in different positions with the probe's orientation directed at the target point while the tip of the probe is maintained at a constant pressure against the tissue surface.

FIG. 6 is a flow chart illustrating the tracking process, according to a second embodiment of the invention.

FIGS. 7A and 7B are perspective illustrations of medical or surgical instruments that may be used in the different embodiments of the invention.

### DETAILED DESCRIPTION OF THE INVENTION

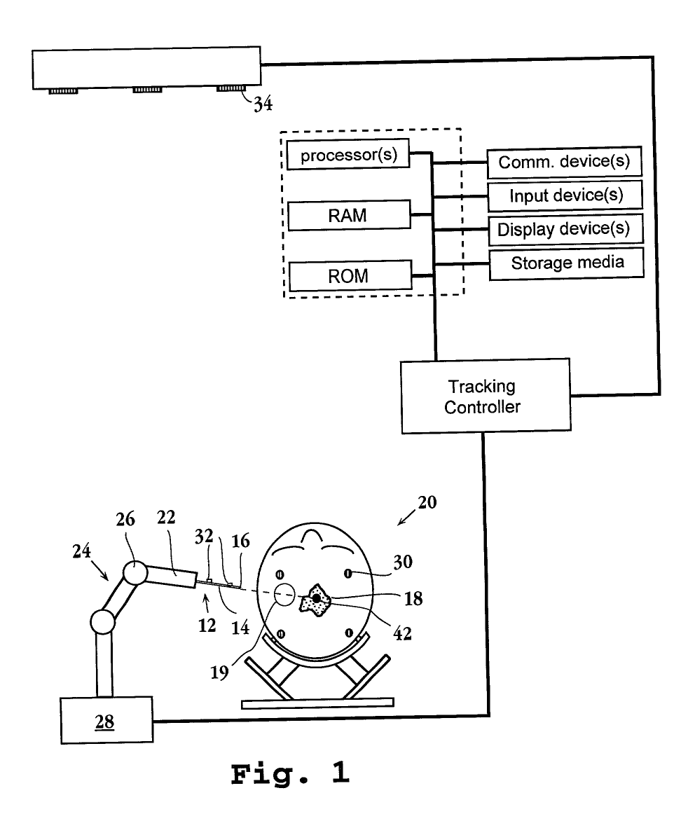

FIG. 1 illustrates an image-guided, robotic-assisted surgery system, which may be used to implement embodiments of the present invention. The system includes a surgical or medical instrument 12 having an elongate axis 14 and a tip 16. In one embodiment, the instrument may be a viewing instrument, such as an endoscope or surgical microscope, equipped with a lens for viewing an internal target site 18 and any intervening tissue 19 of a patient 20. In another embodiment, the instrument is preferably a probe, such as an ultrasound probe for tracking a moving target inside the patient's body. The instrument may also include a pointer or a tool, such as a drill.

In accordance with embodiments of the invention, instrument 12 is releasably attached to the distal-end of an end arm segment 22 of a processor-controlled, motor-driven, multi-arm assembly 24. The assembly is preferably a robotic-arm assembly with one or more fine control motors for precisely controlling movement of the individual arm segments, which are interconnected by universal joints 26 or the like. Typically, there will be one less universal joint than arm segments. The first arm segment of the robotic-arm assembly is attached to a base 28. The robotic-arm assembly may be an articulated arm, a haptic device, or a cobotic device. Descriptions of cobotic devices may be found, for example, in U.S. Pat. No. 5,952,796.

Before the tracking procedures of the present invention are implemented, the patient's target site is registered to images of the site. This may be accomplished in a variety of ways. In one embodiment, a plurality of fiducial markers 30 placed on the patient near the target site are used to register corresponding points on preoperative or intraoperative 2-D image scans of patient target site 18. Corresponding points are those points that represent the same anatomical features in the two spaces.

In general, there are two types of registration image-to-image and image-to-physical. The algorithms employed to accomplish registration are mathematically and algorithmically identical in each case. They use as input the 3-D positions of three or more fiducials in both spaces, and they output the point-for-point mapping from one space to another. The mapping addresses the physical differences in position of the two spaces, which consists of a shift, a rotation, a scale or a combination thereof.

The correct mapping, or registration, is the particular rotation, shift or scale that will map all the localized fiducial positions in one 3-D space, for example, the physical space around the patient in the operating room, to the corresponding localized positions in the second space, for example, a CT image. If these fiducial positions are properly mapped then, unless there is distortion in the images, all non-fiducial points in the first space will be mapped to corresponding points in the second space as well. These non-fiducial points are the anatomical points of interest to the surgeon.

Because of inevitable small errors in the localization of the fiducial points, it is rarely possible to find a rotation, a shift or a scale that will map all fiducial points exactly from one space to the other. Therefore, an algorithm is used that finds the rotation, shift or scale that will produce the smallest fiducial mapping error (in the standard least-squares sense). This mapping error provides a measure of the success of the registration. It is computed by first calculating, for each fiducial, the distance between its localized position in the second space and the localized position in the first space as mapped into the second space. The mapping error is then computed by calculating the square root of the average of the squares of these distances.

In one embodiment, a computer system is used to render and display the 2-D preoperative images and render 3-D volumetric perspective images of target site 18 on a display device. Registration is then accomplished by successively pointing or touching the tip of the instrument to each of the fiducial markers on the patient, moving the computer cursor onto the corresponding image fiducial, and activating an appropriate input device (e.g., clicking a mouse or foot pedal) to map the physical fiducial to the image fiducial. This may be done before or after the instrument is attached to the robot. If done before instrument attachment, instrument 12 will have associated with it a mechanism for tracking the instrument. For example, the instrument can be equipped with a plurality of tracking elements 32 on its shaft 14 which emit signals to sensors 34 positioned in view of the instrument. Both the instrument and the sensors will be in communication with a tracking controller, which is in communication with the computer system that processes the signals received by sensors 34 in carrying out the registration process.

Alternatively, registration may be done with the instrument attached to the robot, since the robot is in two-way communication with the tracking controller.

As previously noted, the registration procedure described above is merely one way of carrying out the registration process. Other ways known in the art may also be employed.

During the surgical procedure, with the instrument attached to the robot, the instrument's position and orientation is known with respect to the robot's coordinate system. Thus, by processing the signals received from the robot through the tracking controller, the computer system is able to track the movement of instrument 12. The instrument may also be tracked using tracking elements 32.

The tracking controller may be a separate element or it may be physically integrated with the computer system and may even be embodied in an option card which is inserted into an available card slot in the computer.

Various aspects of the image-guided, robotic-assisted surgery procedure, including tracking, control of the robotic-arm assembly to enforce a desired orientation of the instrument, and image rendering, may be implemented by a program of instructions (e.g., software) based on initial user input which may be supplied by various input devices such as a keyboard and mouse. Software implementing one or more of the various aspects of the present invention may be written to run with existing software used for image-guided surgery.

The software for such tasks may be fetched by a processor, such as a central processing unit (CPU), from random-access memory (RAM) for execution. Other processors may also be used in conjunction with the CPU such as a graphics chip for rendering images. The software may be stored in read-only memory (ROM) on the computer system and transferred to RAM when in use. Alternatively, the software may be transferred to RAM, or transferred directly to the appropriate processor for execution, from ROM, or through a storage medium such as a disk drive, or through a communications device such as a modem or network interface. More broadly, the software may be conveyed by any medium that is readable by the processor. Such media may include, for example, various magnetic media such as disks or tapes, various optical media such as compact disks, as well as various communication paths throughout the electromagnetic spectrum including infrared signals, signals transmitted through a network or the internet, and carrier waves encoded to transmit the software.

As an alternative to software implementation, the above-described aspects of the invention may be implemented with functionally equivalent hardware using discrete components, application specific integrated circuits (ASICs), digital signal processing circuits, or the like. Such hardware may be physically integrated with the computer processor(s) or may be a separate device which may be embodied on a computer card that can be inserted into an available card slot in the computer.

Thus, the above-mentioned aspects of the invention can be implemented using software, hardware, or combination thereof. The disclosure provides the functional information one skilled in the art would require to implement a system to perform the functions required, with software, functionally equivalent hardware, or a combination thereof.

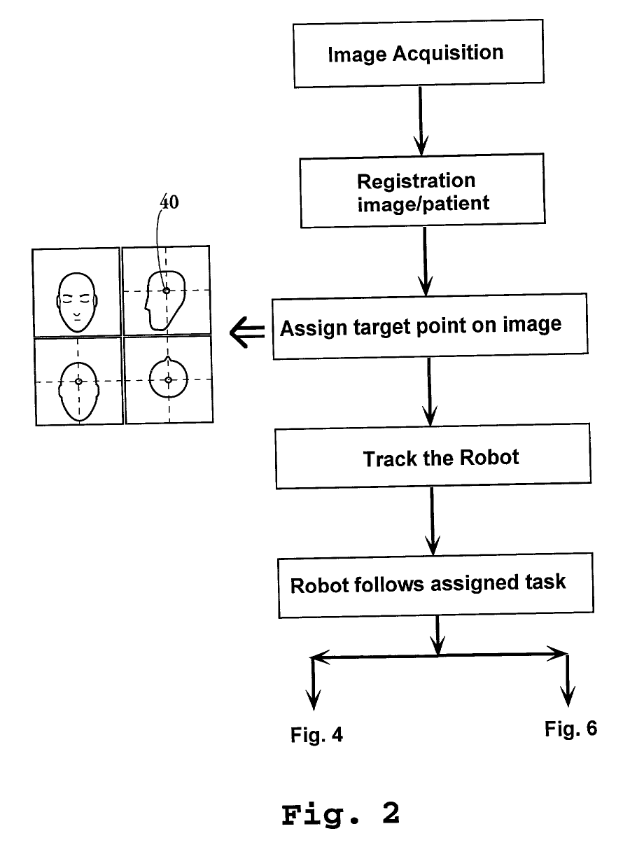

FIG. 2 is a flow chart illustrating the process of setting up the robotic tracking in accordance with embodiments of the invention. First, the preoperative or intraoperative scan data representing internal scans of the patient target site are acquired and used to construct various 2-D images taken in different planes and a 3-D image of the patient target site. These images are displayed on the display device for viewing by the user. The user then assigns an “image” target point 40 on the 2-D images by, for example, pointing the computer cursor at the desired location on the images and inputting information to the computer (e.g., by clicking a mouse or foot pedal) to establish that point as the image target point. The computer establishes a correspondence between assigned target point 40 and a target point 42 in the patient's body by, for example, using point-to-point mapping as is done in the registration procedure. Point-to-point mapping essentially involves determining a transformation matrix that maps the coordinates of point 42 to another set of coordinates representing point 40. The computer stores the target point coordinate data in a storage media, such RAM, ROM or disk. Next, the robot is tracked, as the predetermined task is carried out by the robot.

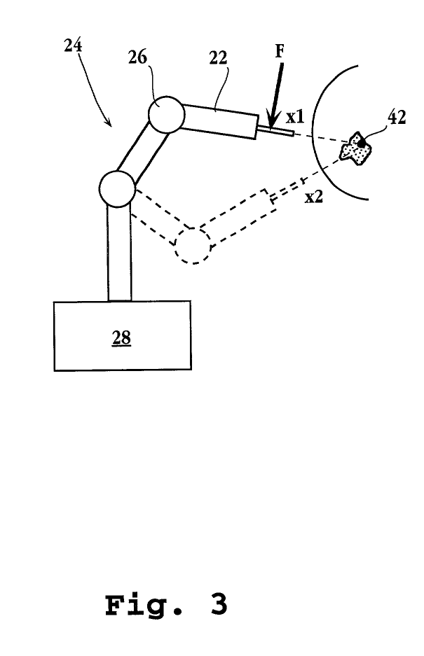

In the first embodiment, the task of the robot is to make the necessary adjustments to keep the viewing instrument directed toward the target point, as the surgeon moves the instrument in space to determine the optimal point of entry to the target site within the patient's body. For example, as the surgeon grasps the end segment 22 and applies a force (F) to it to move the tip of the instrument from point x₁ to point x₂, as shown in FIG. 3, the computer determines the appropriate correction to be applied, and the tracking controller sends signals to the robot to activate its internal motors to move one or more of the arm segments to reorient the axis of the instrument toward the direction of target point 42. This correction, while not instantaneous, is made as the surgeon moves the end arm segment to quasi-continuously maintain colinearity between the axis of the instrument and target point 42.

The instrument is a medical instrument, such as a viewing instrument (e.g., an endoscope) adapted to generate image signals indicative of the view along the axis of the instrument and to transmit such signals to the tracking controller which, in turn, sends the signals to the computer system which processes the signals and renders on the display an image of the patient's target site and any intervening tissue, as viewed along the axis of the instrument.

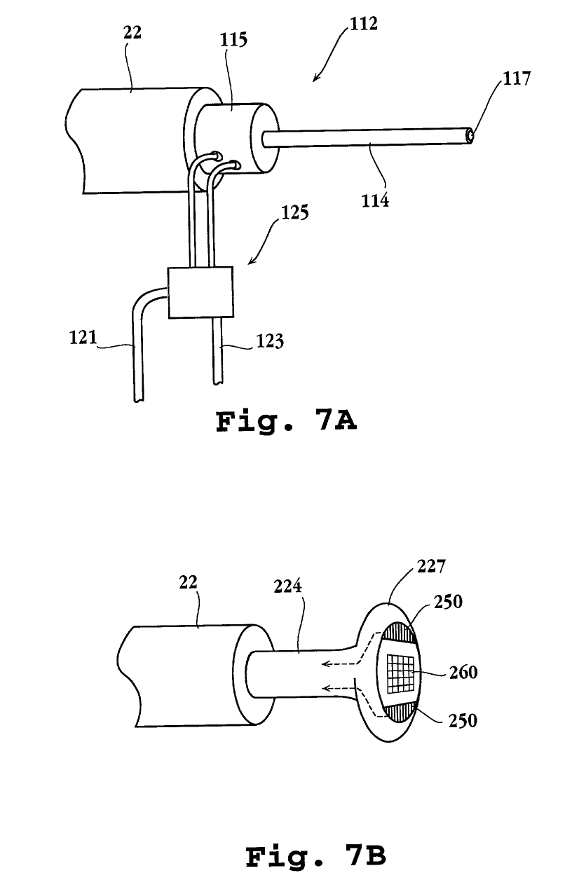

An exemplary endoscope is illustrated in FIG. 7A. The endoscope 112 has an elongate axis 114 and a base 115 that fits into an appropriately sized bore in the distal end of end arm segment 22. The base contains circuitry to transmit images captured by the endoscope through its lens 117. A fiber optic cable 121 and a video cable 123 interface with the endoscope through an adapter 125 to transmit signals to the tracking controller and on to the computer system, as is known in the art.

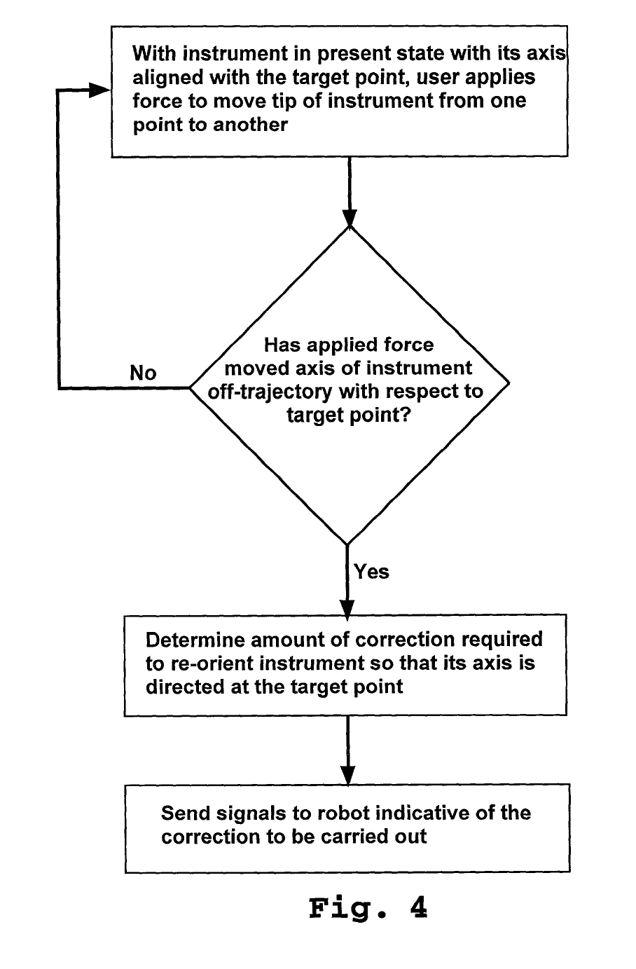

FIG. 4 is a flow chart showing the interactive robot correction process according to the first embodiment of the invention. With the instrument in a present state with its axis aligned with the target point, a user applies a force either to the instrument itself or to the end arm segment of the robot to move the tip of the instrument from one point to another. The computer determines if the applied force has moved the axis of the instrument off-trajectory with respect to the target point and also determines the appropriate correction required by analyzing the signals received from the robot indicative of the position and orientation of the instrument and comparing this data with the target point coordinate data stored in memory. The tracking controller, who is in continuous two-way communication with the computer, then sends signals to the robot to activate its motors to carry out the correction.

In accordance with a second embodiment, the medical instrument is a surgical tool that has a pressure sensor/transducer or the like in the tip of the tool. The tool is preferably an ultrasonic probe, for example, as shown in FIG. 7B. The ultrasound probe has an elongate portion 224, one end of which fits in a bore in the distal end of end arm segment 22. The other end of the probe terminates in a head 227 that has pressure or force contact sensors 250 positioned therein. The sensors are positioned so that the contact surface of the transdu are approximately flush with the contact surface of the probe head. As schematically shown in FIG. 7B, the sensors are in communication with the processor circuitry that controls robotic assembly 24 to provide a feedback signal indicative of the pressure or contact between the probe and a tissue surface. The probe further includes an image array 260 that tracks a moving target in its field of view. Appropriate communication paths may be provided so that the images obtained by the image array may be processed by the computer system and displayed.

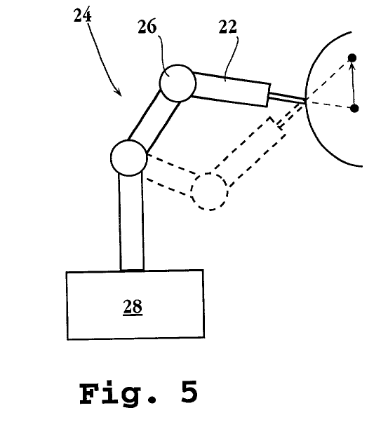

This second embodiment is similar to the first embodiment in that the probe's orientation is enforced along the axis of the probe toward the target point. Here, however, the surgeon does not move the probe; instead, the robot applies the only driving force on the probe to track a moving target, such as the tip of a biopsy needle, inside the body, while the tip of the probe is maintained at a substantially constant pressure against a tissue surface. The tip of the probe is fixed, and the robot is actuated to move the proximal end of the end arm segment to maintain colinearity between the axis of the probe and the target point, as the target moves. Simultaneously, the pressure sensor(s) in the probe tip provide feedback signals to the robot in order to maintain the substantially constant pressure between the probe and tissue surface. During the entire targeting and scanning procedure, the position and the pressure of the probe tip remains constant, as illustrated in FIG. 5. As is the case with the correction in the previous embodiment, this correction, while not instantaneous, is made on a real-time basis.

The target can be tracked via a 3-D localizer or through image processing, i.e., viewing the target on an image.

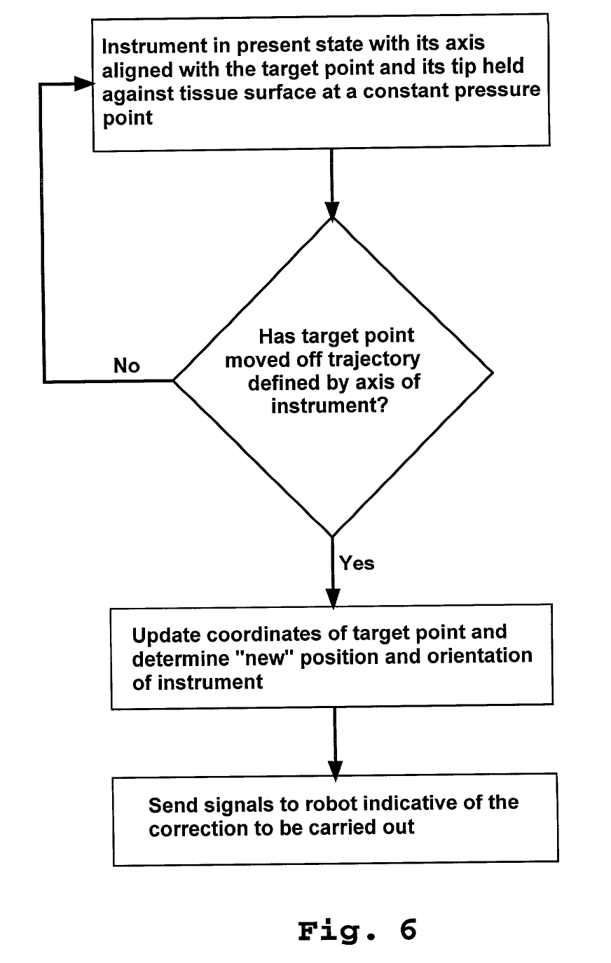

FIG. 6 is a flow chart illustrating the tracking process according to the second embodiment of the invention. With the probe in an initial state with its axis aligned with the target point and its tip held against a tissue surface at a constant, predetermined pressure, the target point moves within the patient's body. As this occurs, the computer updates the coordinates of the target point, determines if the axis of the probe is off-trajectory with respect to the “new” target point coordinates, and determines the appropriate correction required by comparing the “present” position and orientation of the instrument data with the updated target point coordinate data. The tracking controller, who is in continuous communication with the computer, then sends signals to the robot to carry out the correction. While this correction is being carried out, the pressure transducer in the probe tip is also sending feedback signals to the robot to maintain the predetermined pressure between the tissue surface and the probe tip.

This embodiment has various applications. For example, the ultrasonic probe may be used to track a point (e.g., the tip) of a moving biopsy, as it is approaching a targeted lesion inside the body.

While embodiments of the invention have been described, it will be apparent to those skilled in the art in light of the foregoing description that many further alternatives, modifications and variations are possible. The invention described herein is intended to embrace all such alternatives, modifications and variations as may fall within the spirit and scope of the appended claims.

What is claimed:

1. A device for determining the optimal point of entry of a surgical tool adapted for use by a surgeon in accessing a target site within a patient's body, comprising:
(a) an articulated mechanical arm having or accommodating a distal-end pointer;
(b) a tracking controller for tracking the position and orientation of the pointer with respect to a predetermined target coordinate;
(c) an imaging device in communication with the tracking controller for generating an image of the target site and intervening tissue as seen from a selected point outside of the body, along a line between that point and the target point coordinate; and
(d) an actuator, in communication with the tracking controller, for adjusting the position of the mechanical arm so as to orient the axis of the pointer in the direction of the target point coordinate, as the pointer is moved in space to a selected position outside the body;
wherein the user can approach the target site, or view the target site and intervening tissue, along a trajectory from the selected position to the target point coordinate.

2. The device of claim 1, wherein the imaging device constructs an image of the target site using previously obtained scan data, and wherein the predetermined target coordinate is assigned using the constructed image.

3. The device of claim 1, wherein the mechanical arm is a multi-segmented arm.

4. The device of claim 1, wherein, once the optimal point of entry is determined, the pointer can be replaced with a surgical tool to enter the patient's target site along the established trajectory.

5. A method for maintaining a trajectory toward a target site and for viewing any intervening tissue along the trajectory, as defined by the axis of a viewing instrument and a target coordinate in the target site, while the instrument is moved in space, comprising:
(a) acquiring scans of the patient;
(b) using the acquired scans to construct an image of the patient target site;
(c) assigning the target coordinate on the constructed image;
(d) correlating an image coordinate system with an instrument coordinate system; and
(e) controlling the orientation of the instrument to maintain the defined trajectory, as the instrument is moved in space outside the body.

6. A processor-readable medium embodying a program of instructions for execution by a processor to perform a method of maintaining a trajectory toward a target site, as defined by the axis of a viewing instrument and a target coordinate in the target site, while the instrument is moved in space, the program of instructions comprising instructions for:
(a) acquiring scans of the patient;
(b) using the acquired scans to construct an image of the patient target site;
(c) assigning the target coordinate on the constructed image;
(d) correlating an image coordinate system with an instrument coordinate system; and
(e) controlling the orientation of the instrument to maintain the defined trajectory, as the instrument is moved in space outside the body.

7. A device for maintaining a trajectory between a tip of an instrument and a moving target in a patient's body, comprising:
(a) an articulated mechanical arm having or accommodating a distal-end instrument having a tip that has or accommodates a force contact sensor;
(b) a tracking mechanism for tracking the position and orientation of the instrument with respect to coordinates of the moving target;
(c) a processor in communication with the tracking mechanism for calculating and updating the coordinates of the moving target; and
(d) an actuator, in communication with the tracking mechanism, for adjusting the orientation of the mechanical arm, while maintaining a constant pressure between the instrument tip and a surface of the body, so as to maintain the trajectory between the tip of the instrument in the direction of the moving target.

8. A method for maintaining a trajectory between a tip of an instrument and a moving target in a patient's body using a robot-held instrument, comprising:
(a) acquiring scans of the patient;
(b) using the acquired scans to construct an image of the patient target site;
(c) assigning the target coordinate on the constructed image; and
(d) controlling the orientation of the instrument to maintain a trajectory defined by the axis of the probe and a point on the moving target, while maintaining the tip of the instrument at a fixed location against a tissue surface at a constant pressure, as the instrument is moved in space outside the body.

9. A processor-readable medium embodying a program of instructions for execution by a processor to perform a method of maintaining a trajectory between a tip of an instrument and a moving target in a patient's body using a robot-held instrument, the program of instructions comprising instructions for:
(a) acquiring scans of the patient;
(b) using the acquired scans to construct an image of the patient target site;
(c) assigning the target coordinate on the constructed image; and
(d) controlling the orientation of the instrument to maintain a trajectory defined by the axis of the probe and a point on the moving target, while maintaining the tip of the instrument at a fixed location against a tissue surface at a constant pressure, as the instrument is moved in space outside the body.
